(19) United States
(12) Patent Application Publication
(10) Pub. No.: US 2016/0061711 A1
(43) Pub. Date: Mar. 3, 2016

(54) APPARATUS AND METHODS FOR CELLULAR ANALYSIS

(71) Applicant: Chiranjit Deka, Morrisville, NC (US)

(72) Inventor: Chiranjit Deka, Morrisville, NC (US)

(21) Appl. No.: 14/888,771

(22) PCT Filed: May 9, 2014
(86) PCT No.: PCT/US14/37508

§ 371 Date: Nov. 3, 2015

Related U.S. Application Data
(60) Provisional application No. 61/822,593, filed on May 13, 2013.

(51) Int. Cl. G01N 15/14; G01N 33/53; G01N 33/569

(52) U.S. Cl. CPC G01N 15/1459; G01N 33/56972; G01N 33/56966; G01N 33/5304; G01N 15/1436; G01N 15/1425; G01N 15/1404; G01N 2015/1486; G01N 2015/008

(57) ABSTRACT

Disclosed are apparatus and methods for analyzing bodily fluids, such as blood samples, using an integrated hematology analyzer and flow cytometer system. Under the present approach, an integrated system may operate as a closed fluidic system or an open fluidic system, and may selectively perform automated hematologic protocols, flow cytometer protocols, and custom protocols. Such apparatus may, for example, identify and enumerate multiple cell types in whole blood based on cellular morphology, analyze cellular immunoassays using antibodies labeled to cells, and also detect low abundant analytes in whole blood as well as serum and other bodily fluids not attached to cells using bead-based immunoassay methods. The system may include a fluid handling system to control sample flow, an optical transducer that includes a flow cell, optical detectors for light scatter and/or fluorescence, and also an illumination source.

### CROSS REFERENCE TO RELATED APPLICATIONS

This application claims the benefit of U.S. Provisional Application No. 61/822,593, filed May 13, 2013, the contents of which are incorporate by reference in their entirety.

### STATEMENT REGARDING GOVERNMENT SUPPORT

None.

### FIELD

The present approach relates to apparatus and methods for analyzing cells and particles.

### BACKGROUND

Conventional hematology instruments are capable of differentiating and enumerating red blood cells, platelets and the five major sub-populations of leukocytes (white cells) in a human blood sample, namely, the lymphocyte, monocyte, neutrophil, eosinophil and basophil sub-populations. Such instruments commonly operate by first lysing the erythrocytes (red cells) in one aliquot of whole blood sample, and then causing the remaining leukocytes in this aliquot of sample to flow, substantially one-at-a-time, through a narrow aperture or cell interrogation zone while subjecting each cell to a combination of electrical and light energy. A second aliquot of whole blood is diluted and the red blood cells stabilized before causing the said red cells in this diluted sample to flow, substantially one-at-a-time, through a narrow aperture or cell interrogation zone while subjecting each cell to a combination of electrical and light energy. In both cases, while passing through the interrogation zone, a combination of measurements are made to determine each leukocyte's unique characteristics in terms of light scatter, Coulter DC volume, radio frequency (RF) electrical conductivity, polarization, and/or fluorescence. The above measurements allow different types of cells to be identified and counted based on their respective size, shape and internal structures.

Several automated hematology analyzers are available in the market, including Beckman Coulter's GENS™, STKS™, and MAXM™ Hematology Instruments; Abbott Laboratories' Cell Dyne 3000/4000 Hematology Instruments; and Toa's Sysmex Series of Hematology Instruments. In automatically acquiring data on each cell type, all of the above-mentioned hematology instruments use at least two discrete cell-analyzing transducers. One (or more) of these transducers operates to acquires data useful in differentiating and enumerating the five different types of white cells, and another transducer is dedicated to counting and sizing of red cells, white cells and platelets in a precise volume of sample. The respective outputs of the multiple transducers are processed by a central processing unit to provide an integrated cell analysis report. In the Beckman Coulter instruments, an electro-optical flow cell (transducer) produces signals indicative of the respective volume (V), electrical conductivity (C) and light scattering (S) properties of each white cell passing there through to provide a “five-part differential” of the five white cell types. Additional transducers operate on the well known Coulter Principle, one serving to count red cells and platelets in a highly diluted sample, and others serve to count white cells in a lysed sample. Information from the three transducers is processed and, in some cases, correlated (e.g., by multiplying the relative percentage of each white cell subset, as obtained from the electro-optical flow cell, by the absolute number of white cells counted by the Coulter transducer) to provide information about each cell type or subset, e.g., the concentration (number per unit volume) of each white cell subset in the whole blood sample being analyzed. In the Abbott instruments, the five-part differential information is provided by an optical flow cell that detects only light scatter and light polarization information. In the Toa instruments, the five-part differential information is provided by a pair of electrical flow cells (Coulter transducers) that measure only the cell's DC volume and RF conductivity. Different lysing reagents are used to differentially process two or more aliquots of the blood sample, prior to passage through the two transducers. A third Coulter transducer operates to detect and count red cells and platelets. As in the Beckman Coulter and Abbott instruments, the respective outputs of the several transducers are correlated to provide the five-part differential information.

The above instruments employ a number of different strategies for selection of the sensor configurations to differentiate all five sub-populations of the white blood cells. In U.S. Pat. No. 5,125,737, Rodriguez et al. describe DC volume measurements, light scatter measurements within certain relatively broad angular ranges between 10 degrees and 70 degrees, an additional measurement parameter termed “opacity” to achieve five-part different of leukocytes. Rodriguez et al. define opacity as the ratio of a cell's DC impedance (volume) to its RF conductivity. The Beckman Coulter approach to integrate DC and RF measurements within an optical flow cell requires the flow channels to be very narrow (typically approximately 50-60 micron) and the length of this narrow channel also to be very short (approximately 60 micron). These requirements are critical to obtain adequate signal-to-noise ratio in the measurement. However, this approach is complicated to implement because of difficulty of manufacturing the flow cell in an optical grade material which can chip and crack during the manufacturing process.

Others have attempted to perform leukocyte differential by eliminating the need for RF. For example, U.S. Pat. No. 6,232,125 to Deka et al. describes a method for identifying five different populations of leukocytes using DC and light scatter measurements at five different angular ranges, 1-3 degrees, 4-6 degrees, 6-8 degrees, 9-12 degrees and 20-40 degrees. While this method eliminates the requirement to use RF, the requirement for DC measurement does not eliminate the difficulty in manufacturing the flow cells for at least the reasons described above.

The H*1 Hematology Analyzer manufactured by Technicon, Inc., employed a two-step chemical process for differential analysis of leukocytes. First, it provides a four part differential minus basophils. Next, it provides a result for basophils by differentially lysing the other leukocyte sub-populations. Obviously, the time needed for two sequential chemical processes and the cost of additional reagents are disadvantageous. Still another approach is disclosed by Hubi et al. [J. Clin. Lab. Anal. 10:177-183 (1996)] where basophils are identified by using double staining with fluorescence-labeled monoclonal antibodies. Other special methods, such as staining of heparin within the basophils at low pH and in the presence of lanthanum ions, have also been used [Gilbert et. al., Blood, 46:279-286 (1975)] to resolve basophils. As suggested, all of the prior art approaches are relatively complex and expensive to implement.

The eosinophil sub-population of leukocytes also requires special attention in providing a 5-part differential analysis. In some measurement schemes, eosinophils tend to “look like” neutrophils (i.e., in parameter space). The above-noted Terstappen et al. article also discloses the use of orthogonal depolarized light scatter and orthogonal total light scatter intensities to resolve eosinophils from the neutrophils. This method is based on an observation that the refractile granules in the eosinophils tend to induce a greater depolarization of the scattered light in the orthogonal direction. Since this depolarization effect is stronger for the eosinophils than the neutrophils, a scattergram obtained by comparing depolarized orthogonal light scatter with total orthogonal light scatter intensity resolves the eosinophils as a cluster separate from the neutrophils. The method of Terstappen et al. has been used subsequently by Marshal to resolve the eosinophil population in whole blood, as disclosed in U.S. Pat. No. 5,510,267 to Marshall. However, it is generally known that the polarization effect of light scatter is more subtle than angular dependence of total light scatter intensity. Therefore, in general, the detection system required to measure depolarization must be more sensitive, and often more expensive, than that required for discerning angular variation of total light scatter intensity.

Estimation of the number of white cells, red cells and platelets per unit of whole blood volume blood and their associated parameters by an automated hematology analyzer described above is called a complete blood count or “CBC”. The CBC count may be used to find the cause of symptoms such as fatigue, weakness, fever, bruising, or weight loss. It may also be used to identify anemia, measure blood loss, diagnose polycythemia, determine presence of infection, diagnose diseases such as leukemia, check how the body is dealing with some types of drug or radiation treatment, or check effect of abnormal bleeding on the blood cells and counts, as a few examples. Some hematology analyzers offer early indications if there is leukemia or lymphoma present. However, automated hematology analyzers provide no means to pinpoint differences between specific types of lymphoma, such as T-cell lymphoma or B-cell lymphoma. Similarly, if there is indication of infection in a patient sample, conventional hematology analyzers cannot identify the specific type of infection present. These limitations are generally due to two primary facts: (1) conventional hematology analyzers cannot measure differences between cells based on immunophenotypes, and (2) conventional hematology analyzers also cannot measure any component of whole blood that is smaller than platelets, such as many immunologically significant biomarkers that can be found in blood or other bodily fluid, including proteins such as antibodies or antigens that are not attached to cells. As a result, conventional hematology instrument systems are limited to routine blood testing.

Conventional flow cytometers, on the other hand, are capable of differentiating and enumerating cells and biomarkers based on their respective immunological phenotypes. In order to make such measurements, a sample, such as whole blood sample or serum or any other bodily fluid, is first incubated with fluorescently labeled antibodies or antigens (or antibody or antigen conjugated microspheres) called probes. The said probes then bind to specific cells or biomarkers that are in the sample. The labeled cells or biomarkers in the sample are then allowed to flow, substantially one-at-a-time, through a narrow aperture or cell interrogation zone while subjecting each cell to a combination of electrical and light energy. While passing through the interrogation zone, a combination of measurements are made to determine each cell or biomarker's unique immunological characteristics or phenotype in terms of fluorescence signal emitted by the specific probes that are now attached to the said cell or biomarker. For example, an important test performed by a flow cytometer is the CD4 counting test used to monitor the immune status of HIV infected patients. In this test, a whole blood sample is incubated with a reagent containing fluorescently labeled antibodies to a specific type of protein receptors called the CD4 receptor on the surface of certain lymphocytes (a type of white blood cells). The lymphocytes that express these receptors are called CD4 cells. By causing the said labeled blood sample to flow through the flow cell of the flow cytometer, cells labeled with the said CD4 probes are detected and counted as a percent of total lymphocyte count in the sample. A second reference method, usually performed on a hematology instrument system, allows one to measure the absolute number of lymphocytes per microliter of whole blood. Combining the two measurements, one can determine the absolute number of CD4 cells per microliter of whole blood. When this count falls below 400, antiretroviral therapy is initiated. Therefore, ability to count CD4 cells is valuable in managing the health of HIV patients.

Conventional flow cytometers are also capable of identifying and quantifying various biomarkers in a blood sample that are not attached to any cell. This is accomplished by mixing the blood sample with microspheres that have specific capture molecules for the target biomarker, such as antibodies or antigens, pre-attached to their surfaces. When microspheres contact a target biomarker in the sample, the latter is bound to the microsphere. A secondary fluorescently labeled antibody or antigen, also contained in the reagent, then attaches to the captured target biomarker, producing what is known as a sandwich immunoassay on a bead, or a bead-based immunoassay. When the sample is then run through the interrogation zone of the flow cell of a flow cytometer, beads that have captured the said target biomarker produce specific florescence signal, thus identifying the presence of the said biomarker in the sample. For example, C-reactive protein (CRP) is a biomarker whose concentration increases as a result of certain inflammatory processes in the body. It is often used as an indication of risks for cardiac diseases. CRP can be measured using the above mentioned bead-based immunoassay method for detection of biomarker using a conventional flow cytometer.

CD4 and CRP testing are only two of many important diagnostic applications that can be performed on a conventional flow cytometer, but not on a conventional hematology instrument. For example, tests for Malaria and Dengue fever can be performed on a conventional flow cytometer, but not on a conventional hematology instrument.

Although powerful as a tool for measuring immunologically significant cells and biomarkers, conventional flow cytometers, however, are not able to perform a complete blood count as the conventional hematology instruments does. As a result, and because flow cytometers are also expensive, their use as a diagnostic laboratory equipment have been limited so far to mostly the large reference laboratories and flow cytometry core facilities. Because most of the small and medium sized diagnostic laboratories focus on routine blood testing due to the high number of routine tests that are prescribed, those laboratories often have to send out their samples to other larger reference laboratories or specialty flow cytometry core laboratories if immunological testing on conventional flow cytometers is required. This extends the time required to obtain the diagnosis, thereby delaying therapy. It also increases the cost of the diagnosis. Neither outcome is desirable.

As discussed in U.S. Pat. No. 6,228,652 to Rodriguez et al., previous attempts at combining hematology analysis and flow cytometry involved merely connecting a stand-alone flow cytometer instrument and one or more stand-alone hematology instruments into an integrated laboratory testing system in which blood samples are automatically advanced along a track past these different individual instruments. As sample-containing vials pass each instrument, a blood sample is aspirated from each vial and analyzed by the instrument independently of each other. Laboratory systems combining discrete hematology and flow cytometry instruments are commercially available from Beckman Coulter and Toa Medical Electronics, reference being made to Toa's HST Series. This approach is feasible only for the large laboratories in places where cost or space are of not much concern. For small and medium sized laboratories, particularly in resource limited areas of the world, this is not a practical approach.

U.S. Pat. No. 5,631,165 to Chupp et al. describes an approach to integrate the respective functions of hematology and flow cytometry instruments into a single instrument. The disclosed instrument comprises a plurality of transducers, including an optical flow cell adapted to make fluorescence and multi-angle light scatter measurements for white cells, an electrical impedance-measuring transducer (a Coulter transducer) for red cells, and a colorimeter for measuring the overall hemoglobin content of a blood sample. The respective outputs from these transducers are processed and correlated to produce a report on red, white and fluorescent cells.

There are a number of disadvantages to the system described by Chupp et al. As suggested above, the requirement to correlate the respective outputs of multiple transducers in order to report certain characteristics of a cell type or subset can, under certain circumstances, can be problematic in that it introduces an uncertainty in the analytical results. The validity of the requisite correlation step presupposes that the sample processed by one transducer is identical in content to the sample processed by the other transducer(s). This may not always be the case. Ideally, all of the measurements made on a cell should be made simultaneously by the same transducer. In such a case, there would be no need to correlate data from independent or separate transducers. Further, the simultaneous measurement of multiple parameters on a single cell using a single transducer enables a multidimensional cell analysis that would not be possible using separate transducers, or even using a single transducer when the parameter measurements are spatially separated in time.

In addition, Chupp et al. fails to mimic the operation of a conventional flow cytometer. Typically, conventional semi-automatic flow cytometers, which are gold standards in tests such as CD4 cell counting for HIV patients, utilize samples prepared off line by the operator either manually or using automated sample preparation modules that are available in the market. This approach allows the user the freedom to introduce any new method and assay into a flow cytometer as the flow cytometer is not limiting this step. Methods for preparing samples for immunoassays depend on the application and/or disease condition selected for testing. The majority of flow cytometry applications begin with the cells in whole blood that are incubated with reagents containing fluorescently labeled antibodies (i.e., cellular immunoassays), or mixing a blood or serum sample with microspheres that have capture molecules on their surfaces (i.e., bead-based biomarker detection assay). The incubation time and temperature for each assay may be different. The approach described in U.S. Pat. No. 5,631,165 fails to achieve the versatility of semi-automated conventional flow cytometers. Additionally, U.S. Pat. No. 5,631,165 does not teach a method to perform tests for non-cellular analytes or biomarkers in blood using beads as the solid phase. From an operational perspective, the closed fluidic operation of the flow cytometer method described in U.S. Pat. No. 5,631,165 limits the throughput of the system, such as, for example, by holding up the instrument for incubating whole blood with antibodies within the instrument. These and other disadvantages limit the usefulness of the system described by Chupp et al.

U.S. Pat. No. 6,228,652 to Rodriguez et al. describes a specialized flow cell with a substantially rectangular aperture to measure coulter impedance or DC, conductance or RF, light scatter and fluorescence. There are a number of disadvantages to this system. For example, the approach is based on utilizing the hematology flow cell from GenS™ and StakS™ hematology analyzers to also measure one channel of fluorescence. The need to be able to measure DC and RF in an optical flow cell, however, puts significant restriction on both the length and inner dimension (width) of the aperture of this flow cell. Typically, in a conventional flow cytometer the inner dimension of the flow cell is 100 to 200 micron. In contrast, as discussed in in U.S. Pat. No. 6,228,652, in order to get adequate signal to noise in the DC and RF measurements, Rodriguez et al. needed to use a flow cell whose inner width was only 50 micron and length was 65 micron. In order to insert the electrodes that apply the high voltages across the aperture in this flow cell for DC and RF measurements, the inner bore of the flow cell had to be drilled out carefully to within the said 65 micron distance. To make drilling possible, the flow cell must have a substantial thickness to its external walls and must be made of sturdy materials that can withstand mechanical drilling into its inner bore from both ends and to within about 50 to 65 micron of each other. As the flow must be made from optically transparent materials, such as quartz or glass, this process is very delicate because it is prone to chipping and cracking the inside of the flow cell, which then renders it unusable. Unless extreme care is taken, such failures significantly reduce the yield and make such flow cells prohibitively expensive for most prospective users. In addition, the requirement to have a very short length between the electrodes also imposes a restriction on the external length of the flow cell, which in turn makes it difficult to place other optical components close to the flow cell due to mechanical interference. Further, the very narrow aperture makes the flow cell more susceptible to clogging and need for more challenging maintenance.

U.S. Pat. No. 7,611,849 Hansen et al. describes a method for measuring CD4 cells on a hematology analyzer using gold nanoparticles conjugated to anti-CD4 antibodies instead of fluorescent probes. When these particles bind to CD4 cells, their light scatter profile changes due to the attached gold nanoparticles, thus showing the CD4 cells as a different cluster when viewed on the light scatter bivariate plot. However, gold nanoparticles tend to clump together when stored for a significant duration. This degrades the performance of the reagent over time, causing inaccuracies in the results. Additionally, gold nanoparticle based immunoassay reagents for flow cytometers are unable to accurately measure biomarkers that are not attached to any cells. Moreover, in abnormal samples, light scatter patterns of the cells can change significantly relative to the patterns for normal samples. The addition of the further light scatter changes due to the nanoparticles further complicates the analysis in such samples. It is due to the fact that in the above described method by Hansen et al., the immunological and hematological measurements are not independent variables. For the purpose of accuracy, precision and for the measurement approach to be applicable universally, it is highly desirable to use as the primary differentiating parameters for the immunological and the hematology signatures of a cell or biomarker that are mutually independent variables. Therefore, using light scatter signals for both of the above measurements presents numerous disadvantages.

In view of the foregoing discussion, therefore, there is a need for an improved method and apparatus for producing a hematological analysis (i.e., complete blood count) including multi-part differential analysis of leukocytes in a whole blood sample and immunological analysis of cells and biomarkers (i.e, immunoassay) on a single and affordable platform.

There is also a need for an instrument that combines conventional hematology and flow cytometry in a single instrument with a design that substantially retains the flexibility and scalability of a conventional flow cytometer, whereby any number of cellular and biomarker immunoassays can be introduced by the user (i.e., an open system), even as it delivers sample-to-answer hematology results same as conventional hematology analyzers, but without the requiring complex measurements such as DC and RF.

### SUMMARY

In view of the foregoing discussion, there exists a need for a laboratory to be able to analyze blood samples to obtain results that include routine testing results such as complete blood count and advanced testing results such as immunoassays at the cellular as well as biomarker level on a single instrument and more specifically, perform on a single instrument tests that a hematology instrument and a flow cytometer can deliver together.

It is therefore an object of the present approach to provide methods and apparatus for producing an automated sample-to-answer complete blood count, including multi-part differential analysis of leukocytes in a whole blood sample and capable of producing measurements based on immunological characteristics of cells and biomarkers using a flow cell, that do not require the use of DC and RF measurements, and provide for both a closed fluidic system for specific hematological assay protocols as well as an open fluidic system that does not limit the types of immunoassays that can be performed by the user.

Another object of the present approach is to provide instruments that, in combination, are more powerful than an individual hematology analyzer or an individual flow cytometer, but remain simple to operate and easy to maintain. Such apparatus may be especially advantageous to small and medium laboratories.

Another object of the present approach is to provide apparatus featuring an integrated hematology analyzer with flow cytometry capabilities. Such apparatus may, for example, analyze cellular immunoassays using antibodies labeled to cells, and also detect low abundant analytes in whole blood as well as serum and other bodily fluids not attached to cells using bead-based immunoassay methods.

It is another object of the present approach to provide methods and apparatus in which immunological measurements can use fluorescently labeled probes and do not require the use metallic nanoparticles based probes to enhance light scatter signals of the target cells as a means to identify their immunological phenotypes.

It is yet another object of the present approach to provide the user a graphical user interface to operate the apparatus as either an automated hematology instrument or as a flow cytometer.

Under the present approach, an integrated hematology analyzer and flow cytometer system may include an optical flow cell having a flow cell body with a flow channel and a through hole in the flow cell body configured to allow light propagating along an axis substantially perpendicular to the flow channel, to illuminate the flow channel, among other features. The system may include a plurality of light scatter detectors arranged to detect light scattered by constituents of a sample flowing through the flow channel at a plurality of detection angles relative to the axis. The system may include a fluorescent light optical lens system to detect fluorescent light emitted by constituents of a sample flowing through the flow channel in a direction substantially orthogonal to the axis. The system may include a fluid handling system to direct a sample from a sample vessel to other components of the system, such as the flow cell, based on a selected protocol from a set of defined protocols. The defined protocols can include hematologic protocols, flow cytometer protocols, and/or custom protocols, and the system may include reagents and mixing capabilities for sample preparation according to a selected protocol. The system may also include a controller to configure and operate the fluid handling system according to the selected protocol.

An optical transducer for an integrated hematology analyzer and flow cytometer apparatus may be used in the present approach. Such a transducer may include an optical flow cell having a flow cell body, a flow channel housed within the flow cell body and having a first end and a second end, a sample insertion tube in fluid connection with the first end of the flow channel, a sheath fluid insertion tube in fluid connection with the first end of the flow channel, a through hole in the flow cell body configured to allow light propagating along an axis substantially perpendicular to the flow channel, to illuminate the flow channel, and a waste removal tube in fluid connection with the second end of the flow channel, among other features. The optical transducer may also include a plurality of light scatter detectors arranged to detect light scattered by constituents of a sample flowing through the flow channel at a plurality of detection angles relative to the axis. For example, the detection angles may include a first angle of about 1° to about 2°, a second angle of about 9° to about 12°, and third angle of about 25° to about 45°. The optical transducer may also include a fluorescent light optical lens system to detect fluorescent light emitted from constituents of a sample flowing through the flow channel in a direction substantially orthogonal to the axis. The optical lens system may include a plurality of optical filters, a plurality of fluorescence detectors, and at least one lens.

According to embodiments of the present approach, a multi-part differential analysis of the white blood cell (leukocyte) population in a whole blood sample may be attained by: (a) lysing the red blood cells with a lytic reagent; (b) causing the lysed sample to flow into a flow channel; and (c) producing a plurality of signals (for example, in a four signal embodiment, LS1-LS4) from the remaining leukocytes respectively representing the light-scattering properties of such leukocytes within different angular ranges (for example, in a four signal embodiment, ANG1-ANG4). In some embodiments, three of such angular ranges may be lower than 40 degrees, measured with respect to the direction of propagation of an illuminating light beam. In some embodiments, at least one additional angular range may be substantially orthogonal to the direction of propagation of the illuminating light beam. In some embodiments, the angular ranges lower than 45 degrees may be, as examples, ANG1=about 1 to about 2 degrees; ANG2=about 9 to about 12 degrees; and ANG3=about 25 to about 45 degrees. In some embodiments, the orthogonal angular ranges may be ANG4=about 75 to about 105. Further, immunoassays may be attained by aspirating a sample of blood or serum, which may be pre-labeled with fluorescent probes, and producing a plurality of fluorescence signals representing the abundance of immunological markers on a cell or concentration of specific biomarkers in the sample.

In some embodiments, multi-part differential analyses of leukocytes may be attained by: (a) producing a plurality of first electrical signals proportional to intensities of lights scattered by said individual leukocytes within different angular ranges (for example, in a three angular range embodiment, LS1-LS3); (b) producing a second electrical signal proportional to intensities of axial light loss; (c) producing a third electrical signal proportional to fluorescence intensities of dye molecules incorporated in the individual leukocytes; and (d) differentiating and enumerating the sub-populations of leukocytes based on comparison of said first, second and third electrical signals.

According to embodiments of the present approach, immunological measurements may be attained by: (a) labeling cells and/or biomarkers in a sample with fluorescent probes; (b) causing the labeled sample to flow through a flow cell; and (c) producing a plurality of fluorescence signals. The signals in some embodiments may be excited by an illumination source emitting electromagnetic radiation, for example radiation in the red wavelength range of the visible spectrum, and as another example, in the wavelength range of about 630 nm-650 nm. In some embodiments the illumination source is a diode laser. In another embodiments, the illumination source may be a laser, such as a laser emitting in the wavelength range of about 470-540 nm, for example. In other embodiments, two lasers may be used, to emit a plurality of fluorescence signals, for example, in the wavelength range of about 500-780 nm.

Methods of the present approach may be carried out in an apparatus comprising an optical flow cell. An optical flow cell may include a flow channel, though which blood cells or particles are caused to flow seriatim and allowed to pass through a substantially focused zone of electromagnetic energy, hereinafter referred to as the interrogation zone. Response of the cells and particles to electromagnetic radiation may be detected by various electromagnetic energy detectors placed in desired locations around the optical flow cell.

Embodiments of the apparatus may further include: (a) means for causing cells and particles in the sample to pass through the interrogation zone seriatim, such as a pump; (b) means for illuminating individual blood cells and particles passing through the interrogation zone with at least one beam of light propagating along an axis, each illuminated cell and particles acting to scatter light incident thereon and producing fluorescence if labeled with fluorescent probes; (c) means for detecting the intensity of light scattered from an illuminated blood cell in the interrogation zone, within predetermined angular ranges, such as the plurality of different angular ranges described above and below; (d) means for detecting the intensity of fluorescence light from an illuminated blood cell in the interrogation zone, within the predetermined wavelength ranges, such as the plurality of different wavelength ranges already described; and (e) means for differentiating red blood cells, platelets and five major sub-populations of leukocytes in the sample, such as, for example, lymphocyte, monocyte, neutrophil, etc., based on the respective amplitudes of the respective electrical signals produced by detecting scattered light. Embodiments of the apparatus and methods used therein may include means for enhancing the measured differentiation between different leukocyte sub populations based on fluorescence signal from dyes bound to the said leukocyte sub populations. Embodiments of the apparatus may also include means for differentiating the immunologically significant blood cells or biomarkers based on the respective amplitudes of the respective fluorescence signals produced by detecting fluorescent light intensities.

In some embodiments, the apparatus may feature a fluidic system with a first fluidic module for hematological complete blood count analysis, and a second fluidic module for immunological measurements. The first fluidic module may cause the apparatus to perform: (a) aspiration of whole blood from a sample tube, (b) segmenting two separate aliquots of the aspirated whole blood sample, (c) lysing the red cells in one aliquot by mixing the sample with a lytic reagent, (d) causing the remaining white cells to flow though a flow cell and into a waste reservoir, (e) adding a diluents solution to the second aliquot of whole blood, and (f) causing the diluted whole blood sample though a flow cell and into a waste reservoir. The second fluidic module may cause the apparatus to perform: (a) aspirating a blood or serum sample previously exposed off-line with immunologically specific reagents labeled with fluorescent molecules, and (b) causing the said sample to flow through a flow cell and into the waste reservoir. In some embodiments of the present approach, the two fluidic modules share the same flow cell. Each fluidic module may be operated independently of the other, such as by, for example, a user selectable software switch.

The present approach will be better understood from the ensuing detailed description of preferred embodiments, reference being made to the accompanying drawings.

### DESCRIPTION OF THE DRAWINGS

FIGS. 1(a)-1(e) are block diagrams showing embodiments of methods for operating (a) a hematology analyzer; (b) a flow cytometer; (c) an integrated hematology analyzer and flow cytometer; (d) an integrated instrument as a hematology analyzer; and (e) an integrated instrument as a flow cytometer.

FIG. 2 is a block diagram of an embodiment of a method for preparing a sample in an integrated apparatus operating as a hematology analyzer.

FIG. 3(a) is a schematic of an embodiment of a flow cell with a capillary tube as a flow channel. FIG. 3(b) is a schematic of an embodiment of a flow cell with a cuvette tube having a square cross section, as a flow channel.

FIG. 4 is a diagram of an embodiment of a flow cell with a laser beam, multi-angle light scatter detector array, and fluorescence detectors.

FIG. 5 is a diagram of an embodiment of an apparatus with a flow cell and a fluidic module.

FIG. 6 is a schematic showing the operation of the embodiment shown in FIG. 5 bypassing the hematology sample preparation fluidics to perform the workflow of a flow cytometer.

FIG. 7 is a depiction of a demonstrative graphical user interface according to an embodiment of the present approach.

FIG. 8(a) shows a demonstrative diagram showing different populations of leukocytes resolved by measuring light scatter at different angles using an apparatus embodying the present approach, in hematology mode. FIG. 8(b) shows a demonstrative diagram showing platelets, mature Red Blood Cells and Reticulocytes identified and enumerated by comparing light scatter and fluorescence signals using an apparatus embodying the present approach, in hematology mode.

FIG. 9 shows an example bivariate plot of fluorescently labeled cells generated using an apparatus embodying the present approach, running the workflow as a flow cytometer.

FIG. 10 shows example results from a bead-based assay for a biomarker generated using an apparatus embodying the present approach.

### DETAILED DESCRIPTION

The present approach allows for an all-optical measurement platform that combines the capability of an automated multi-part hematology analysis platform with the power and versatility of a flow cytometry platform within a single, low-cost, easy to use apparatus. Using a single flow cell transducer in conjunction with modular fluidic sub-systems that may perform the work flow of a hematology analyzer and/or a flow cytometer, apparatus embodying the present approach may be used as a closed system in which whole blood sample is used to produce a set of pre-programmed protocols to produce a pre-determined set of diagnostic parameters on whole blood. Alternatively, apparatus embodying the present approach may be used as an open system, like a conventional flow cytometer, in which any bodily fluid, including but not limited to blood, serum, plasma, and urine, can be used to analyze immunological characteristics of targeted cells, pathogens or biomarkers in the bodily fluid.

In some embodiments, the apparatus may be used as stand-alone instrument analyzing one tube of sample manually presented to it by the user at a time. In some embodiments, the instrument may be used in a high throughput setting, such as a reference laboratory, by integrating the apparatus with an automated conveyor belt or carrousel containing multiple patient samples.

In some embodiments the flow cell of the apparatus may be made of one or more optically transparent capillary tubes, and may be of substantially cylindrical dimensions. In other embodiments the flow cell of the apparatus may be made of one or more optically transparent capillary tubes, and may be of substantially square dimensions. In some other embodiments, the apparatus may use a flow cell made from a prism, such as a cuvette tube, and may have, for example, a square or rectangular cross section.

In some embodiments, some or all reagents necessary to perform one or more assays may be contained on-board the apparatus. In some embodiments, the apparatus may be connected to vessels containing some or all reagents necessary to perform one or more assays.

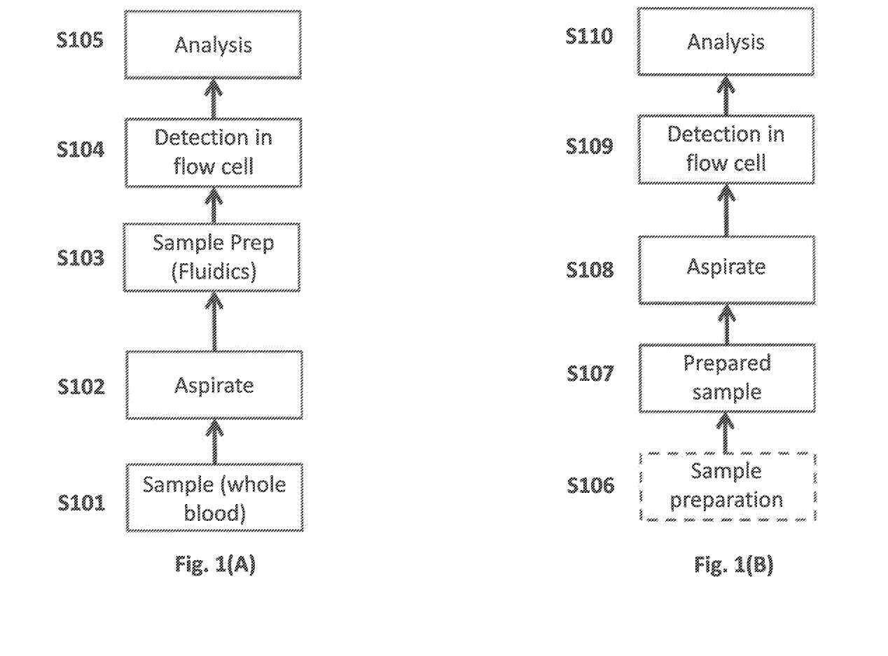

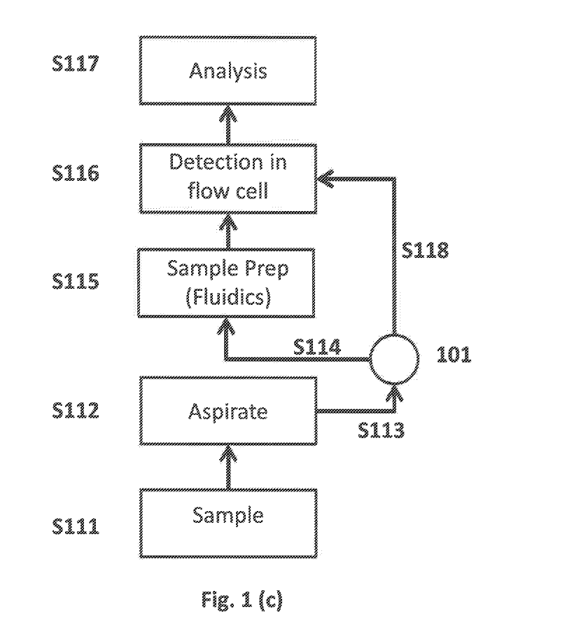

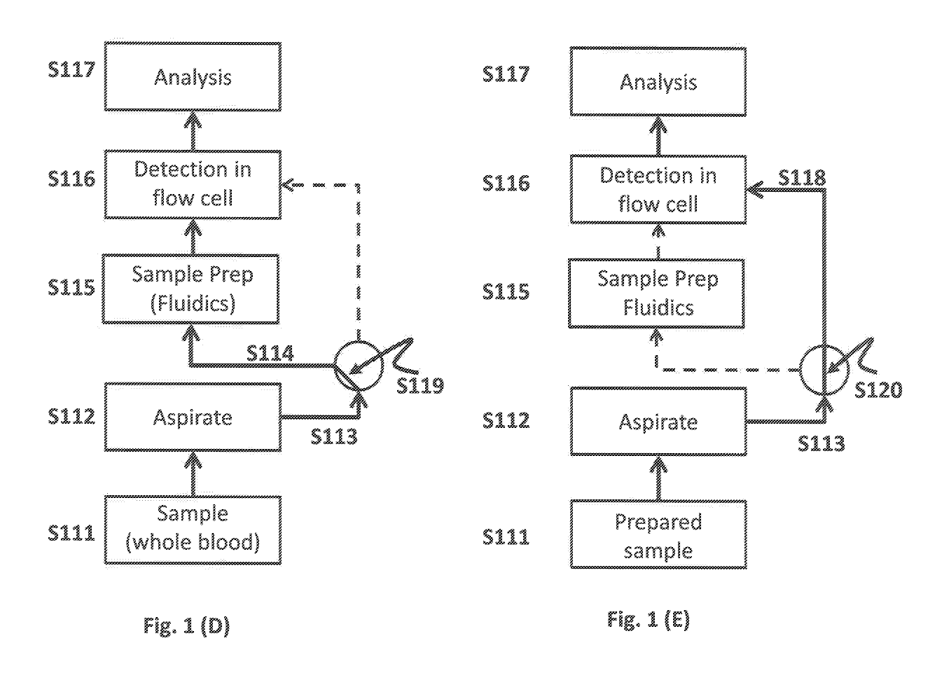

To illustrate the present approach, FIGS. 1(a) and 1(b) show the work flow for automated hematology analysis and flow cytometry, respectively. A controller may be incorporated in an embodiment to control components, such as components in a fluid handling system that may include fluid flow direction devices such as valves and pumps, to achieve the desired workflow. As shown in FIG. 1(a), in an automated hematology analyzer work flow, whole blood may be presented to the instrument in a sample tube S101, which aspirates S102 a pre-determined volume of the blood using an aspirating tube or needle. Alternatively, a volume sample may be aspirated over a predetermined period of time. A controller (the same controller for the fluid handling system, a separate controller, or a combination of controllers) may be programmed to control aspiration. The sample is then processed S103 in an automated sample preparation fluidic module. After processed sample then detected and measured S104 in a flow cell before being analyzed S105 using an analyzer employing, for example, signal processing electronics and software.

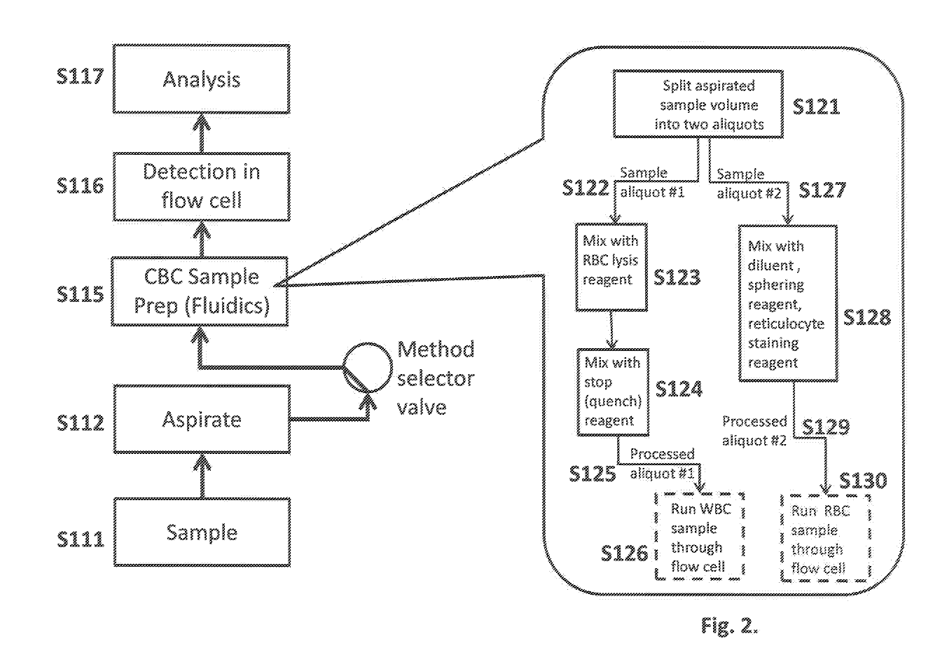

FIG. 2 shows an embodiment of a method for preparing a sample in an integrated apparatus operating as a hematology analyzer. Referring to FIG. 2, the basic sample preparation steps in an automated hematology analyzer may include the splitting of an aspirated blood volume into at least two aliquots S121, a first aliquot or sample aliquot #1, and a second aliquot or sample aliquot #2. Sample aliquot #1 may be directed to a mixing cup S122 where it is mixed with a lytic reagent S123, followed by another solution to stop the lytic reaction, such as a quenching solution S124. The resultant mixture in aliquot #1, now containing intact while blood cells and red cell debris, may then be directed to a flow cell S125, where the contents are hydrodynamically focused to run through the flow cell in seriatim S126. The contents may subsequently be detected by, for example, optical means S116, and analyzed S117 using an analyzer employing, for example, signal processing electronics and software. Sample aliquot #2 may be directed to a mixing cup S127 where it is mixed with reagents S128 that comprises a diluent which may or may not additionally include components that substantially render the red blood cell (RBC) spherical in shape and also a RNA staining fluorescent dye that penetrates the membrane of the RBC to bind to the RNA of the immature RBCs commonly known as the Reticulocytes. The resultant sample mixture may then be directed to a flow cell S129, where the contents are hydro dynamically focused to run through the flow cell in seriatim S130. The contents may subsequently be detected by, for example, optical means S116, and analyzed S117 using an analyzer employing, for example, signal processing electronics and software. Apparatus embodying the present approach may be pre-programmed to operate pursuant to this method for a specific assay, and operate as a closed system or a closed work flow. Embodiments may include a controller for controlling operation of the apparatus, such as the operation of a fluid handling system, to achieve the desired work flow.

The closed system for automated hematology analysis may be useful for ensuring repeatability and precision of results.

FIG. 1(b) shows the work flow of a flow cytometer. Sample preparation S106 may be performed off line, as indicated by the broken lines, and usually comprises reacting a sample with fluorescently labeled antibodies, fluorescently labeled antigens, fluorescently labeled nucleotides, or fluorescent dyes or combinations thereof. The sample may be, as examples, whole blood, blood serum, or any suspension of cells or biomarkers in a bodily fluid or other buffers. A prepared sample may be presented to the aspirating needle of the flow cytometer S107, which aspirates a volume of sample S108, for example, either a pre-determined volume of the sample, or volume of sample for a pre-determined period of time. The aspirated sample is then directed to the flow cell for detection by, for example, optical means S109, which may be subsequently analyzed S110 using an analyzer employing, for example, signal processing electronics and software. This work flow is often referred to as an open system or open work flow, as it is independent of the sample preparation protocols. Featuring an open system in a flow cytometer is useful for many reasons, such as expanding the menu of uses without upgrading or replacing the instrument. It may also be important to small and medium sized laboratories and in resource limited settings, in which instruments are not frequently replaceable due to cost constraints.

In embodiments of the present approach, the two work flows may be integrated by the use of flow-controlling means, such as, for example, flow switches, pumps, and valves (for example, a routing valve). A controller may be used to control operation of the apparatus to achieve the desired work flow, such as, for example, by controlling the operation of switches, pumps, and valves. The switches, pumps, and valves may be part of a fluid handling system incorporated into the embodiment. In some embodiments, the apparatus may use a completely separate fluidic module for flow cytometry and connect the same to the flow cell using a T-section or a Y-section, wherein the other branch of the said T-section or Y-section may be connected to a different fluidic module or assembly independent from the fluidic module for flow cytometry. Referring to FIG. 1(c), a sample may be presented to the aspirating needle or tube S111. The sample may be either whole blood (in case of hematology analysis) or previously prepared flow cytometry sample, depending on the test being conducted. Whole blood or serum or other bodily fluid (sample) may be incubated with fluorescent labels. The aspirating needle or tube aspirates S112 a volume of the sample and drives it S113 into a routing valve 101. The routing valve 101 may direct the sample to the sample preparation fluidic module S114, if the apparatus is to operate in hematology mode, such as an automated hematology analyzer. The automated sample preparation module prepares the sample according to requirements of a selected hematology protocol S115. After sample preparation, the sample may run through a flow cell for detection S116 and analysis S117. Alternatively, if the apparatus is to be operated in flow cytometry mode, for example, the test to be conducted is a flow cytometry immunoassay, then a previously prepared sample (e.g., with immunological probes already attached to target cells or biomarkers) may be aspirated into the routing valve 101, and the routing valve 101 may bypass the hematology sample preparation fluidic module and direct the sample S118 through the flow cell for detection S116 and analysis S117. Note that the steps S116 and S117 may be different for hematology and flow cytometry, depending on, for example, the selected assay and pre-configured operating parameters for the selected assay.

FIG. 1(d) shows the operation of the hematology work flow for the embodiment described in FIG. 1(c), according to one embodiment of the present method. As shown, the flow-controlling means (in this embodiment, the routing valve 101) connects the sample aspiration needle to the sample preparation stations as represented by S113, S119 and S114. The dashed line represents a deactivated or intentionally blocked fluidic channel.

FIG. 1(e) shows the operation of the flow cytometry work flow in the embodiment described in FIG. 1(c), according to one embodiment of the present method. As shown, the flow-controlling means (in this embodiment, the routing valve 101) connects the sample aspiration needle to the flow cell bypassing the sample preparation step S115 as represented by S113, S120 and S118. The dashed lines represent a deactivated or intentionally blocked fluidic channel.

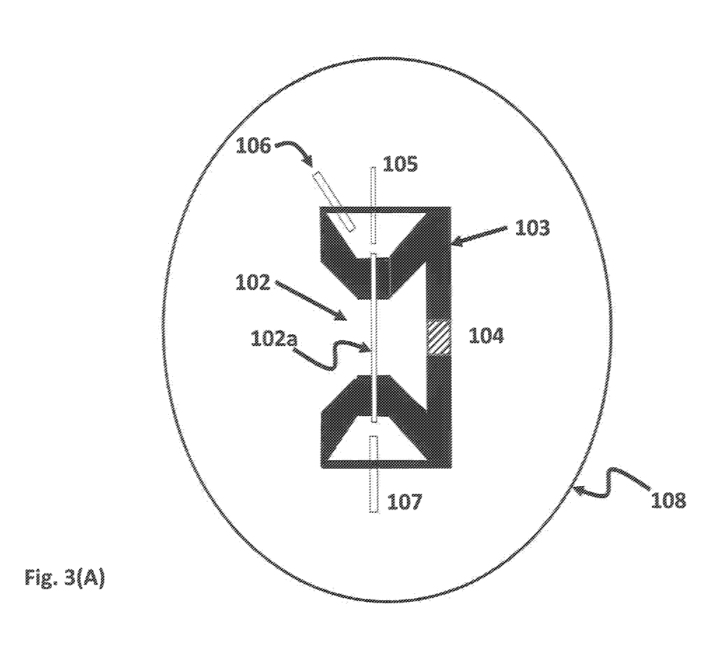

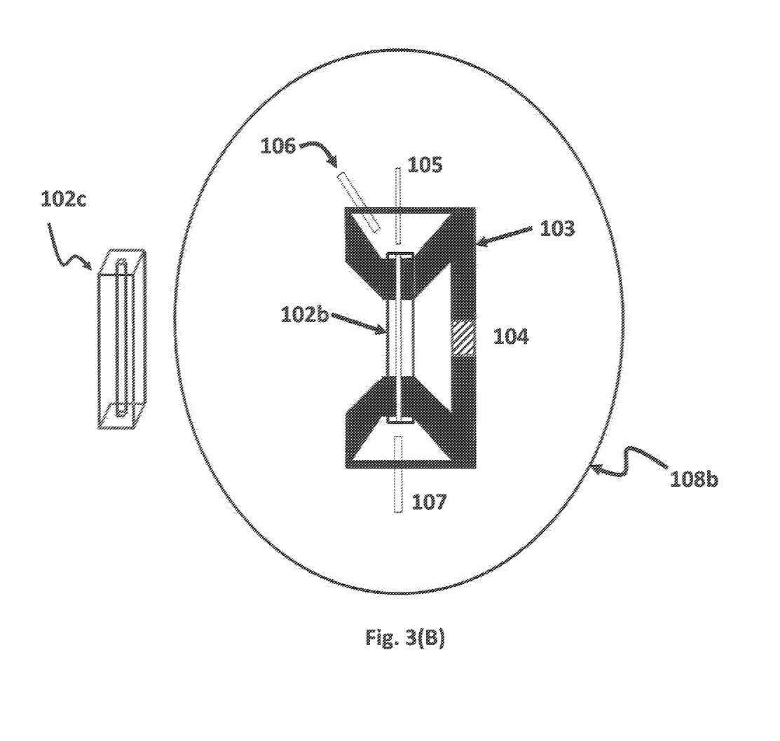

Embodiments of the present approach may feature a single optical transducer that includes the flow cell, optical detectors for light scatter and fluorescence, and an illumination source. The illumination source may also be separate but connectable to the optical transducer. Referring to the embodiment shown in FIG. 3(a), flow cell 108 features a flow channel 102, a flow cell body 103, a sheath fluid insertion tube 106, a waste removal tube 107, and a sample insertion tube 105. The sheath fluid hydrodynamically focuses the fluid stream that flows through the flow channel 102. The insertion tubes 105 and 106 may be fluidly connected to a first end of the flow cell body 103, such that sheath fluid and sample may flow into the flow channel 102, e.g., via pump (not shown). The flow cell body 103 may optionally feature a first void space, such that sheath fluid and sample to flow into the void space at desired flow rates, mix, and then flow into the flow channel 102. The waste removal tube 107 may be fluidly connected to a second end of the flow cell body 103, such that sheath fluid and sample that have flowed through the flow channel 102 may exit the flow cell 108. The flow cell body 103 has a through hole 104 to allow a laser beam to pass through it and intersect the capillary 102. The through hole 104 may be a physical gap in flow cell body 103, or alternatively may be a material that allows light from a source of electromagnetic radiation alternatively referred to as a light source (not shown) to pass through and illuminate the flow channel 102 (in the embodiment shown, flow channel 102 is a capillary tube 102a). In some embodiments, the light source may be one or more lasers, one or more lamps, or one or more light emitting diodes, or any combination thereof. In some preferred embodiments, the laser may be a solid-state laser, a gas laser or a diode laser. In some other embodiments the solid state laser the lasing medium may be pumped by a diode laser, generally known as a diode pumped solid state laser or DPSS.

In some embodiments, the flow channel may be a capillary tube. The capillary tube may be substantially cylindrical, such as a cylinder with an inner diameter equal to or greater than about 75 micron, but less than or equal to about 250 micron, and may have a length greater than about 1 mm. The flow channel may also be a prism. For example, in some embodiments the flow channel may be a flow-through cuvette, such as a cuvette having a square cross section 102b, as shown in FIG. 3(b). Such a cuvette is also represented separately, 102c, on the left side of FIG. 3(b). The signals in some embodiments may be excited by an illumination source emitting electromagnetic radiation, for example radiation in the red wavelength range of the visible spectrum, and as another example, in the wavelength range of about 630nm-650 nm. In some embodiments the illumination source is a diode laser. In another embodiments, the illumination source may be a laser, such as a laser emitting in the wavelength range of about 470-540 nm, for example. In other embodiments, two lasers may be used, to emit a plurality of fluorescence signals, for example, in the wavelength range of about 500-780 nm.

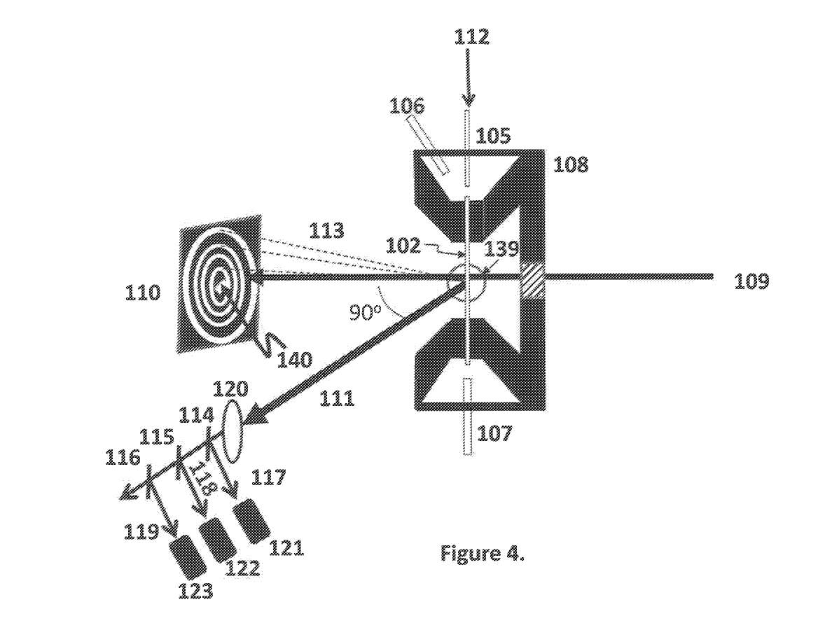

FIG. 4 shows an embodiment of a flow cell, multi-angle light scatter detector array 110, and fluorescence detectors 121, 122, and 123, illuminated by laser beam 109. In one embodiment, shown in FIG. 4, the flow cell 108 is integrated with optical sensors comprising multi-angle light scatter detectors 110. The light scatter detectors 110 may be mounted in a plane perpendicular to the direction of the laser beam. In some embodiments, scattered light may additionally be measured in a direction substantially orthogonal 139 to the laser beam 109. When a cell or particle flowing through the flow channel 102 passes through the laser beam 109, the light is scattered in various directions 113.

The angular distribution of the scattered light 113 depends on the size, shape, internal structure and refractive indices of the said cells or particles. Generally, low angle light scatter provides information that is representative of size, while high angle light scatter, for example 90 degree light scatter, offers information on complexity of the particles. However, such generalization is limited because theoretical calculations have shown that intensity of scattered light for a given particle is represented by an undulating function of the scatter angle. For particles with complex structures, such as white blood cells, the angular distribution is even more complex. As a result, in order to maximize the ability to differentiate between different cell types of substantially similar size, for example various sub populations of white blood cells, embodiments of this invention measures light scatter at several angles as described above. In some embodiments, scattered light 113 may be detected in three angular ranges ANG1, ANG2, and ANG3. The angular ranges may be selected to provide a higher resolution of morphological differences between cells, among other advantageous benefits. For example, in one embodiment, the three angular ranges may be lower than 45 degrees, such as, for example, ANG1=about 1 to about 2 degrees; ANG2=about 9 to about 12 degrees; and ANG3=about 25 to about 45 degrees. In embodiments with an additional detector 139 capable of orthogonal light scatter measurement, it may be useful for providing additional resolution for light scatter signals. The orthogonal angular ranges may be, for example, ANG4=about 75 to about 105. In other embodiments an additional detector 140 placed directly along the axis of the light can be used to measure extinction, also called axial light loss. Axial light loss represents the decrease of the amount of light falling on this detector as a particle or cell passes though the light, casting a momentary shadow that can be representative of the size of the said particle or cell. In one preferred embodiment, axial light loss and light scatter signals at ANG2=about 9 to about 12 degrees; and ANG3=about 25 to about 45 degrees and ANG4=75-90 degrees may be measured.

In some embodiments, in addition to the light scatter detectors 110, the apparatus further includes fluorescence detectors 121, 122, 123. Fluorescence detectors 121, 122, 123 may be in a direction substantially orthogonal to the direction 111 of the laser beam 109 and the direction of flow 112 of the cells or particles in the flow cell. The fluorescent light in this direction 111 may be collected by optical lens system 120 resolved into multiple spectral ranges 117, 118, 119 using optical filters 114, 115, 116. One of ordinary skill would appreciate that an apparatus according to the present method may feature a different number of spectral ranges, optical filters, and angular ranges.

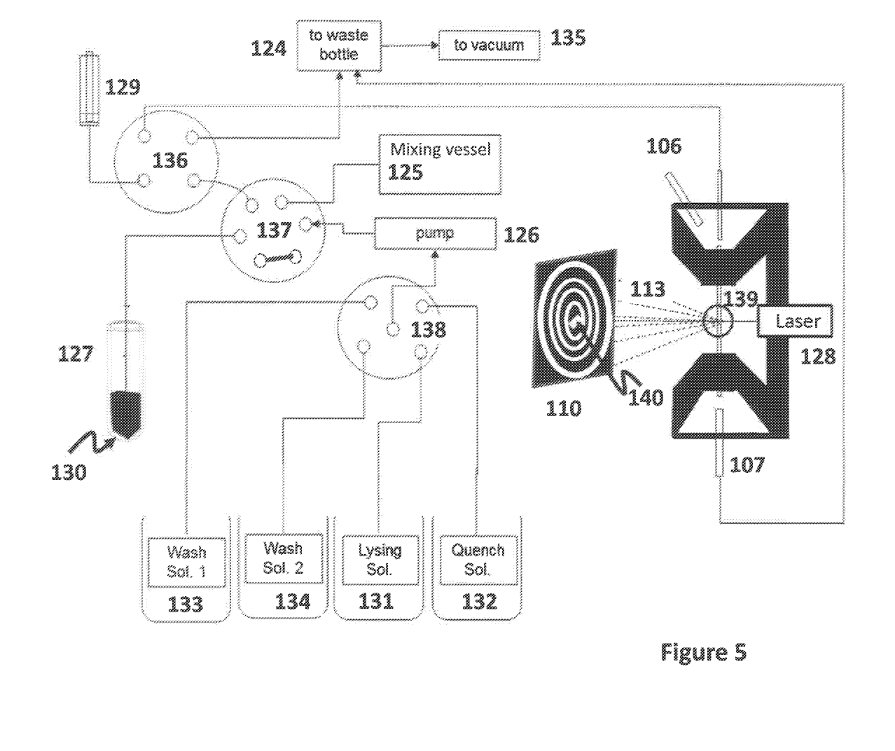

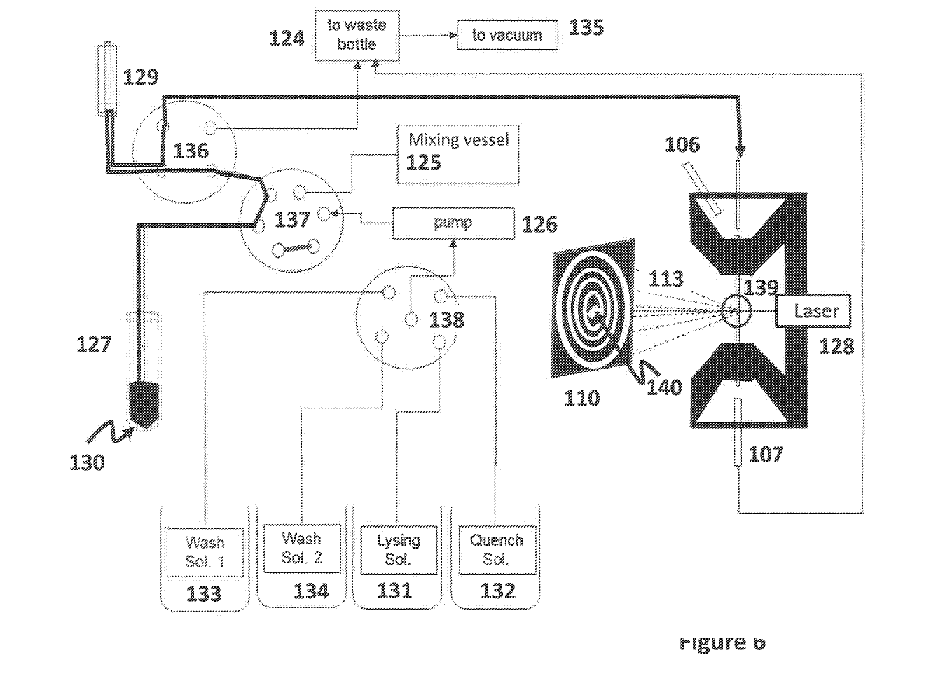

FIG. 5 shows an embodiment according to the present approach in which the flow cell 108 is further integrated a fluidic system used to perform the hematology work flow. The fluidic system depicted in FIG. 5 is demonstrative of a fluid handling system that may be incorporated into an embodiment of the present approach, and may be used to control fluid flow through the embodiment (e.g., volume, direction, rate, etc.), such as to achieve a desired work flow (e.g., open or closed, depending on the desired protocol). In the embodiment shown, the system includes valves 135, 136 and 137, pump 126, syringe pump 129, and mixing vessel 126. These components may be fluidly connected, such that fluid (e.g., a sample) may flow from one component to another without exposure to external conditions, without contamination sourced from outside the components, and/or without leakage or spillage of fluid. Two components in fluid connection may have intermediate components also in fluid connection, such as, for example, two valves in fluid connection may have a pump between the valves (in terms of fluid flow), and in fluid connection with each valve. A fluid handling system may incorporate such components, and a controller may be used to control operation of the fluid handling system or a subset of components, to achieve a desired work flow. Reagents may be included with the system, and may be contained in, for example, different reservoirs 131, 132, 133, and 134. Waste bottle 124 is connected to a vacuum pump 135 and the waste tube 107 of the flow cell. The sheath fluid tube 106 is connected to reservoirs containing sheath fluid and a pump (not shown in this figure). Sample 130 is contained in a sample tube 127. In this embodiment, the fluidic handling system includes valves 135, 136, 137 that may be multi-port valves each of which can be set electronically by a controller to route different fluids in more than one or two different directions or flow paths during a single work flow (using pumps, gravity, and/or other devices to force fluid flow in the desired direction, at the desired rate). In some embodiments, the fluidic system may include valves that route a fluid in only or two directions. In some embodiments, the fluidic system may include valves that are combination of the two different types of valves mentioned above. In yet other embodiments, the fluidic system may comprise fluidic circuits embedded in plastic manifolds. In some embodiments, the fluidic system may comprise microfluidic circuits. In some other embodiments, the microfluidic circuits may utilize droplet based electro-wetting methods to control the flow of fluids. Although not shown in FIG. 5, fluorescence detectors may also be included in the system, in addition to light scatter detectors 110.

FIG. 6 shows an embodiment in which the fluidic system is set to direct the aspirated sample to the flow cell bypassing the sample preparation steps of the hematology operations described in FIG. 5. For instance, the fluid handling system has been set to accomplish the desired configuration (e.g., flow direction(s), volume, and/or rate through one or more components). Some embodiments may feature a fluid handling system controlled by a controller that adjusts the configuration to achieve the desired work flow. In the configuration shown in FIG. 6, fluid flow bypasses the valve 138 and the hematology reagent reservoirs 131-134, as shown by the dark arrow. This embodiment allows the system to be operated as a flow cytometer.

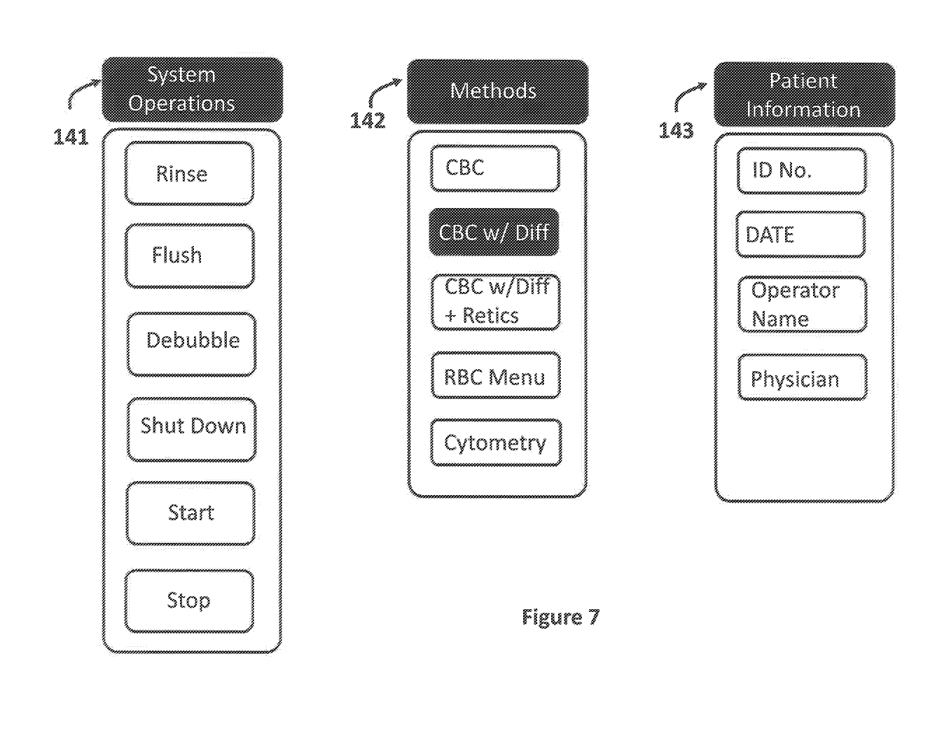

Alternatively, an apparatus according to the present approach can be used as a hematology analyzer and a flow cytometer by selecting a work flow from a Graphical User Interface (GUI). FIG. 7 shows an exemplary embodiment of a GUI, comprising a user activated GUI panels for Systems Operations 141, Methods Selection 142 and Patient (Sample) Information 143. Using the tabs under the Methods Selection 141, specific protocols may be activated, such as for example only, the protocol for Complete Blood Count (CBC) or CBC with five-part leukocyte differential, or the flow cytometry protocol. Similarly, specific systems operations such as rinsing the system fluidics (Rinse) or removal of bubbles in the fluidic lines (Debubble), or shutting down the system (Shut Down) can be activated by selecting each operation manually using the GUI. The GUI may include options for a user to program a custom assay or a custom set of systems operation protocols, such as a custom protocol user interface. A custom protocol user interface may be a GUI that permits a user to define a protocol, such as a hematologic protocol or a flow cytometer protocol. The defined protocol may include a number of defined variables, such as, for example, defined flow direction(s), flow rates, sample volumes, reagent volumes, mixing times, etc., such that the user may instruct one or more controllers operating the fluid handling system with the steps necessary to prepare one or more samples pursuant to the protocol, and also (if desired) direct the sample(s) to a flow chamber for analysis. The custom protocol may include instructions to automate the protocol for multiple samples. Alternatively, the software of the system may be configured such that multiple samples can be run sequentially without user intervention.

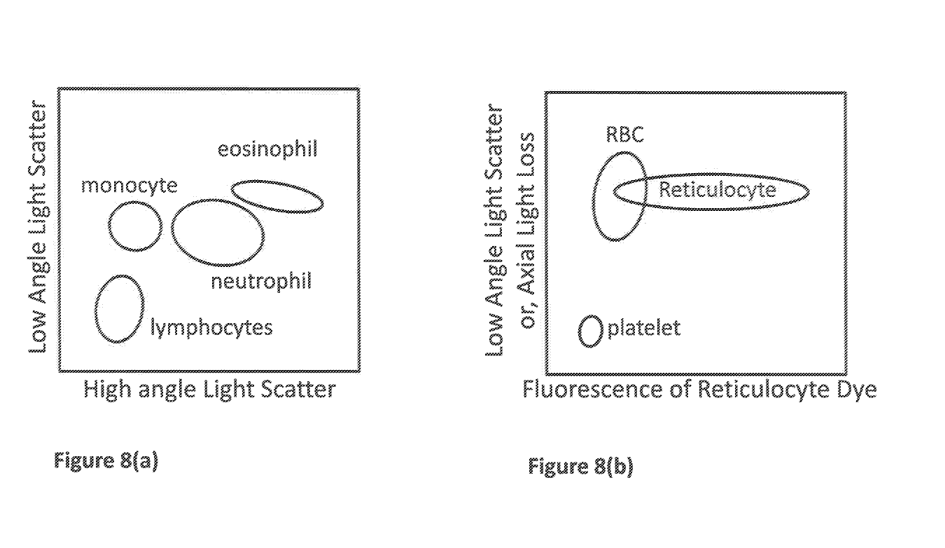

FIG. 8(a) shows a demonstrative diagram of different populations of leukocytes resolved by measuring light scatter at different angles using an apparatus embodying the present approach, in hematology mode. The diagram represents an example of multi-part differential detection and enumeration of the white blood cells using an embodiment of the present approach when the system is run in the hematology operation mode (for example, CBC with five-part differential). The relative arrangement of leukocyte populations in FIG. 8(a) is demonstrative. The actual relative locations and areas for the leukocyte populations in practice may differ from the arrangement as shown. For example, the eosinophil population may be shifted to the upper-right quadrant of the diagram. FIG. 8(b) shows a demonstrative diagram showing platelets, mature Red Blood Cells and Reticulocytes identified and enumerated by comparing light scatter and fluorescence signals using an apparatus embodying the present approach, in hematology mode. The output shown in FIG. 8(b) exemplifies output for the aliquot #2 when an apparatus embodying the present method is operated with the Method Selection set for “CBC w/Diff+Retics”, for example, as shown in FIG. 7. In this protocol, the red blood cells in aliquot #2 is additionally treated with a RNA (ribonucleic acid) specific fluorescence stain and the fluorescence signals from certain stained red blood cells represent the presence of RNA in those cells, indicative of immature of red blood cells also known as Reticulocyte or in short Retics. As with FIG. 8(a), the relative location of the constituents in the diagram may differ in practice.

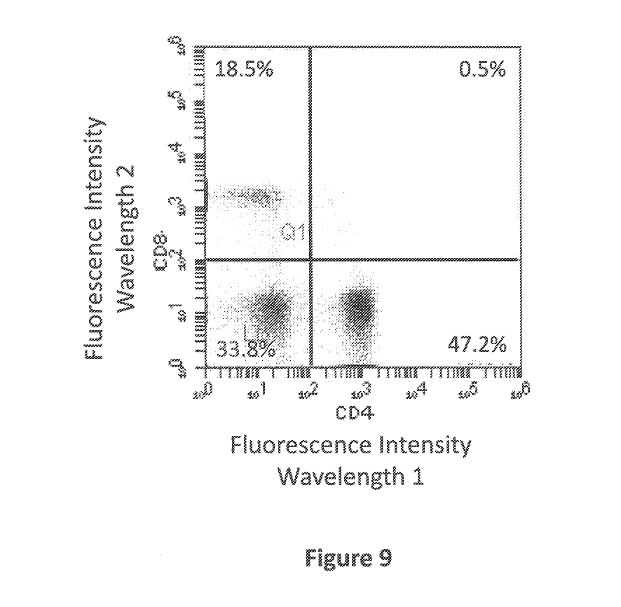

FIG. 9 shows a two dimensional plot comparing fluorescence intensities of cells at two different wavelengths. The present approach may be used to generate such output. To generate the data, a blood sample was exposed to and incubated with anti-CD4 antibodies labeled with a first fluorochrome that emits fluorescence of wavelength 1 and anti-CD4 antibodies labeled with a second fluorochrome that emits fluorescence of wavelength 2. Lymphocytes were selected from a light scattergram, and upon plotting the 2-color fluorescence measurements for all lymphocytes, CD4 and CD8 cells were resolved as separate clusters as show above. As seen in the demonstrative plot of FIG. 9, apparatus embodying the present approach may be used to generate valuable data to accurately resolve cells, such as lymphocytes cells that express the CD4 and CD8 receptors.

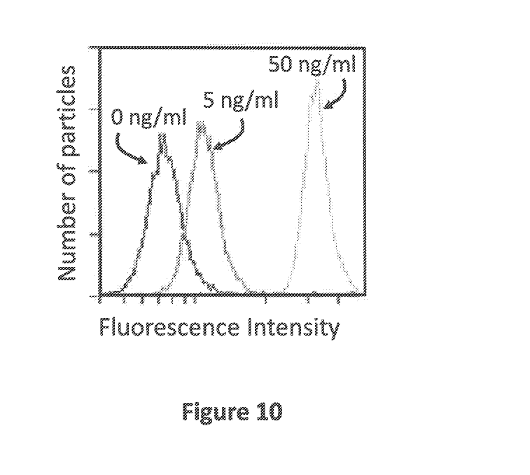

In one embodiment of the present approach, samples containing different concentrations of p24 antigen, a protein associated with the HIV virus, were reacted with polystyrene microspheres having anti-p24 antibodies conjugated on their surfaces. The p24 antigens in the samples bind to the anti-p24 antibodies on the microspheres. Further adding in this reaction mixture a protein that specifically binds to the anti-p24 antibody and labeled with a fluorochrome and running the sample in this apparatus in the flow cytometry work flow, histograms of fluorescence intensities for the microspheres were obtained for each different concentration of the p24, namely 0 ng/ml, 5 ng/ml and 50 ng/ml. FIG. 10 shows example results from this bead-based assay for a biomarker generated using an apparatus embodying the present approach.

The results demonstrate the present approach's ability to detect and quantify biomarkers in a sample. In other embodiments of the present approach, such as depicted in FIG. 4, for example, more than one biomarker can be detected by using microspheres having different capture protein on their surfaces and different corresponding fluorescent probes that emit fluorescence in different wavelengths.

As will be appreciated by one of skill in the art, aspects or portions of the present approach may be embodied as a method, system, and at least in part, on a computer readable medium. Accordingly, the present approach may take the form of a combination an apparatus, with or without reagents, and hardware and software embodiments (including firmware, resident software, micro-code, etc.), or an embodiment combining aspects of an apparatus with software and hardware aspects that may all generally be referred to herein as a “circuit,” “module” or “system.” Furthermore, the present approach may take the form of a computer program product on a computer readable medium having computer-usable program code embodied in the medium. The present approach might also take the form of a combination of such a computer program product with one or more devices, such as a modular sensor brick, systems relating to communications, control, an integrate remote control component, etc.

Any suitable non-transient computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the non-transient computer-readable medium would include the following: a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a device accessed via a network, such as the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any non-transient medium that can contain, store, communicate, propagate, or transport the program for use by or in connection with the instruction execution system, apparatus, or device.

Computer program code for carrying out operations of the present approach may be written in an object oriented programming language such as Java, C++, etc. However, the computer program code for carrying out operations of the present approach may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

The present approach is described below with reference to flowchart illustrations and/or block diagrams of methods, apparatus (systems) and computer program products according to embodiments of the approach. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a non-transient computer-readable memory, including a networked or cloud accessible memory, that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to specially configure it to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

Any prompts associated with the present approach may be presented and responded to via a graphical user interface (GUI) presented on the display of the mobile communications device or the like. Prompts may also be audible, vibrating, etc.

Any flowcharts and block diagrams in the Figures illustrate the architecture, functionality, and operation of possible implementations of systems, methods and computer program products according to various embodiments of the present approach. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems which perform the specified functions or acts, or combinations of special purpose hardware and computer instructions.

The terminology used herein is for the purpose of describing particular embodiments only and is not intended to be limiting of the approach. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The invention may be embodied in other specific forms without departing from the spirit or essential characteristics thereof. The present embodiments are therefore to be considered in all respects as illustrative and not restrictive, the scope of the invention being indicated by the claims of the application rather than by the foregoing description, and all changes which come within the meaning and range of equivalency of the claims are therefore intended to be embraced therein.

1. An optical hematology analyzer and flow cytometer system comprising:
(a) an optical flow cell defining a flow channel;
(b) a light source configured to emit light at the flow channel along an axis substantially perpendicular to the flow channel;
(c) a plurality of light scatter detectors arranged to detect light scattered by constituents of a sample flowing through the flow channel and to provide scattered light optical data, each scattered light detector arranged to detect light scattered at a detection angle relative to the axis, and a detector to measure axial light loss;
(d) a fluorescent light optical lens system configured to detect fluorescent light emitted by constituents of a sample flowing through the flow channel and to provide fluorescent light optical data, the optical lens system comprising a plurality of optical filters, a plurality of fluorescence detectors, and at least one lens;
(e) fluid handling system configured to direct a sample from a sample vessel to the flow cell based on a selected protocol from a set of defined protocols, the set of defined protocols comprising at least one hematologic protocol and at least one flow cytometer protocol;
(f) a controller to configure and operate the fluid handling system according to the selected protocol; and
(g) an analyzer configured to perform at least one of a hematologic protocol and a flow cytometry protocol, based on only optical signal data, wherein optical signal data includes scattered light optical data and fluorescent light optical data.

2. The optical hematology analyzer and flow cytometer system of claim 1, wherein the at least one hematologic protocol comprises a protocol for detection and enumeration of at least one of platelets, red blood cells, and white blood cells.

3. The optical hematology analyzer and flow cytometer system of claim 1, wherein the hematologic protocol comprises a protocol for detection and enumeration of at least three subpopulations of white blood cells.

4. The optical hematology analyzer and flow cytometer system of claim 2, wherein the at least one hematologic protocol comprises at least one of (1) a protocol for detection and enumeration of at least five subpopulations of white blood cells, where in the five populations comprise lymphocytes, monocytes, neutrophils, eosinophils and basophils, and (2) a protocol for detection and enumeration of at least one of red blood cells and platelets.

5. The optical hematology analyzer and flow cytometer system of claim 1, wherein the at least one flow cytometer protocol comprises at least one of an analysis of cell types based on immunological phenotypes; an analysis of non-cellular analytes; and a custom flow cytometer protocol.

6. The optical hematology analyzer and flow cytometer system of claim 1, further comprising a custom protocol creation user interface.

7. The optical hematology analyzer and flow cytometer system of claim 1, wherein the fluid handling system comprises:
(a) a first fluid flow device for aspirating a sample from a sample vessel;
(b) a second fluid flow device for directing the aspirated sample to at least one of a third fluid flow device and the flow cell.

8. The optical hematology analyzer and flow cytometer system of claim 7 wherein the third fluid flow device comprises a plurality of valves and at least one pump, and the system further comprises a controller to configure and operate the fluid handling system to perform at least one of the following:
(a) segment a first volume of an aspirated sample into a first aliquot;
(b) segment a second volume of an aspirated sample into a second aliquot;
(c) direct the first aliquot to a first reaction vessel;
(d) mix the first aliquot with a lysing reagent in the first reaction vessel;
(e) direct at least a portion of the mixture of the first aliquot and lysing reagent from the first reaction vessel to the flow cell;
(f) rinse the first reaction vessel with a rinsing solution and empty the first reaction vessel;
(g) direct the second aliquot to a second reaction vessel;
(h) mix the second aliquot with a diluting reagent;
(i) direct at least a portion of the mixture of the second aliquot and the diluting reagent from the second reaction vessel to the flow cell;
(j) rinse the second reaction vessel with a rinsing solution and empty the second reaction vessel.

9. The optical hematology analyzer and flow cytometer system of claim 8, wherein the fluid handling system is configured to perform at least one protocol selected from the group comprising:
(A) (c) to (f) before (g) to (j);
(B) (c) to (f) substantially in parallel with (g) to (j);
(C) (g) to (j) before (c) to (f);
(D) (c) to (f); and
(E) (g) to (j).

10. The optical hematology analyzer and flow cytometer system of claim 8, further comprising a graphical user interface configured to instruct the controller to configure and operate the fluid handling system to perform a protocol selected from the group comprising:
(A) (c) to (f) before (g) to (j);
(B) (c) to (f) substantially in parallel with (g) to (j);
(C) (g) to (j) before (c) to (f);
(D) (c) to (f); and
(E) (g) to (j).

11. The optical hematology analyzer and flow cytometer system of claim 1 wherein the flow channel comprises one of a capillary tube and a cuvette tube, the tube having an effective internal cross-sectional width of greater than about 75 micron, but less than or equal to about 250 micron.

12. The optical hematology analyzer and flow cytometer system of claim 1, wherein the plurality of light scatter detectors detect light at detection angles are less than 45°.

13. The optical hematology analyzer and flow cytometer system of claim 1, wherein the detection angles comprise a first angle of about 1° to about 2°, a second angle of about 9° to about 12°, and third angle of about 25° to about 45°.

14. (canceled)

15. The optical hematology analyzer and flow cytometer system of claim 1, further comprising an orthogonal light scatter detector arranged to detect light scattered at a generally orthogonal angle relative to the axis.

16. The optical hematology analyzer and flow cytometer system of claim 15, wherein the generally orthogonal angle is about 75° to about 105°.

17. An optical transducer for an optical hematology analyzer and flow cytometer apparatus comprising:
(a) an optical flow cell defining a flow channel;
(b) a plurality of light scatter detectors arranged to detect light scattered by constituents of a sample flowing through the flow channel at a plurality of detection angles relative to an axis substantially perpendicular to the flow channel, and provide scattered light optical data based on the detected scattered light, and a detector to measure axial light loss; and
(c) a fluorescent light optical lens system configured to detect fluorescent light emitted from constituents of a sample flowing through the flow channel and provide fluorescent light optical data;
wherein the transducer is configured to provide optical signal data comprising scattered light optical data and fluorescent light optical data.

18. The optical transducer of claim 17, wherein the flow channel comprises one of a capillary tube and a cuvette tube, the tube having an effective internal cross-sectional width of greater than about 75 micron, but less than or equal to about 250 micron.

19. The optical transducer of claim 17, wherein the plurality of detection angles are less than 45°.

20. The optical transducer of claim 17, wherein the detection angles comprise a first angle of about 1° to about 2°, a second angle of about 9° to about 12°, and third angle of about 25° to about 45°.

21. (canceled)

22. The optical transducer of claim 17, further comprising an orthogonal light scatter detector arranged to detect light scattered from an illuminated flow channel at a generally orthogonal angle relative to a direction of illumination and a direction of fluid flow in the illuminated flow channel.

23. The optical transducer of claim 22, wherein the generally orthogonal angle is about 75° to about 105°.

24-28. (canceled)

29. A method for analyzing a sample on an optical hematology analyzer and flow cytometer apparatus, the method comprising:
a. selecting a hematologic protocol or a flow cytometer protocol;
b. receiving a sample in a sample vessel in fluid connection with a reaction vessel;
c. directing a volume of sample from the sample vessel to the reaction vessel;
d. preparing the sample according to the selected protocol;
e. directing the volume of sample to an optical flow cell defining a flow channel;
f. illuminating at least a portion of the volume of sample in the flow channel along an axis;
g. detecting at least one of scattered light using a plurality of light scatter detectors at a plurality of detection angles relative to the axis and a detector to measure axial light loss, and fluorescent light using a plurality of fluorescence detectors; and
h. generating optical signal data having at least one of scattered light optical data and fluorescent light optical data; and
i. differentiating the constituents of the sample based on only the optical signal data.

30. The method of claim 29, wherein the selected protocol is a hematologic protocol comprising at least one of a leukocyte differential for identifying and enumerating at least three leukocyte subpopulation, leukocyte differential for identifying and enumerating at least five leukocyte subpopulation an RBC protocol for identification and enumeration of red blood cells, a platelet protocol for identification and enumeration of platelets, identification and enumeration of reticulocytes, identification and enumeration of nucleated red blood cell, identification and enumeration of immature blood cells, and a custom hematologic protocol.

31.

32. The method of claim 29, wherein the selected protocol is a cytometer protocol comprising at least one of an analysis of cell types based on immunological phenotypes; an analysis of non-cellular analytes; and a custom flow cytometer protocol.

33. The method of claim 29, wherein the detection angles comprise a first angle of about 1° to about 2°, a second angle of about 9° to about 12°, and third angle of about 25° to about 45°.

34. The method of claim 29, wherein steps b. through i. are repeated for a plurality of samples.
